(19) United States
(12) Patent Application Publication
(10) Pub. No.: US 2009/0092645 A1
(43) Pub. Date: Apr. 9, 2009

(54) Radiation Method for Fabrication of Nano-sized Compound Antibacterial Fabric Textile

(76) Inventors: Te-Hsing Wu, Taoyuan County (TW); Bin Lin, Taoyuan County (TW); Nini-Chen Tsai, Taoyuan County (TW); Chia-Chieh Chen, Taoyuan (TW); Lie-Hang Shen, Taoyuan (TW); Wuu-Jyh Lin, Taoyuan County (TW)

Correspondence Address: MICHAEL LIN, 5F 79 Roosevelt Rd. Sec. 2, TAIPEI, 106, TW

(21) Appl. No.: 11/867,701

(22) Filed: Oct. 5, 2007

(51) Int. Cl. A61K 9/70; A61P 31/04

(52) U.S. Cl. 424/404

(57) ABSTRACT

The invention discloses an innovative process to produce highly antibacterial nano-composite fabric textile containing silver metal compound. The process mainly uses high-energy D-ray radiation to modify silver type of bactericides and graft or crosslink them onto Nylon or PET fiber surface to produce excellent antibacterial textile products. The mechanism uses silver nano-compound as performance additive and through Co-60 irradiation technique reduces and firmly fastens silver particles onto Nylon or PET (Polyethylene Terephthalate) fiber material. Because the inorganic silver type bactericides actively interact with enzymes in bacteria or destroy cell walls to achieve good bactericidal effect, the radiation process does not need initiators or other additives. So the process is simple and effective. The test results prove excellent bactericidal power and potential value in household or medical textile products.

### BACKGROUND OF THE INVENTION

1. Field of the Invention

The invention is related to an innovative radiation method for fabrication of nano-metal compound antibacterial fabric textile. Especially it refers to the process that uses high-energy γ-ray treatment to the surface of Nylon or PET fiber to start the reaction of the silver compound and form the antibacterial nano-composite.

2. Description of the Prior Art

Presently the antibacterial textile products basically use silver nano-particles as antibacterial additive. Due to its excellent thermal stability, it can be added into fiber mother solution. Then after spinning process, antibacterial fiber can be produced. However since the diameter of nano-particles is very small and their surface activity is large, they tend to form aggregates under the high-temperature influence in the process. To stabilize and disperse nano-particles, it requires the use of dispersants. In this way, not only the cost will increase but also the process will become difficult. It is desired to develop a new technology to simplify the manufacturing process that will firmly fasten inorganic antibacterial particles onto textile fibers in the back stage of the process.

Since 1960, radiation technique has been used to effectively make two different polymers into copolymers through grafting or crosslinking mechanism [Hughes, 1973]. This is because when the material surface is exposed to radiation the material becomes in an excited state and produces free radicals and peroxides to initiate grafting and crosslinking reactions. Radiation grafting technique can simply combine two different polymers through radiation. The process is simple and has a great potential to textile industry. Presently, there are examples of using Co-60 radiation to graft acrylic acid, N-isopropylacrylamide or chitosan onto nonwovens [Hao-Tsi Lin, 2003] to improve hydrophilicity, function and bactericidal ability. But the reaction is a little complicated because it requires Co-60 radiation to form membranes of acrylic acid or N-isopropylacrylamide on fibers first, and then uses UV radiation to graft chitosan. The invention is to further simplify radiation technique for antibacterial fabric manufacturing process. The fabric material is commonly used Nylon or PET.

Based on bactericidal principles, the antibacterial materials can be divided into two categories, organic bactericidal powders and inorganic bactericidal powders. In the last century, people developed a variety of organic bactericides to fast and effectively eliminate bacteria [Maxwell and Critchlow, 1997; Subbalakshmi and Sitaram, 1998]. Although organic bactericides have strong bactericidal effect, their biggest shortcoming is bacteria tend to generate resistance to them. Besides, organic bactericides have shortcomings in poor thermal stability and poor chemical stability et al. They are not suitable for radiation grafting technique. This is also why inorganic bactericides receive more attention. Further, inorganic bactericides can be divided into photocatalyst type, like nano titanium dioxide, nano zinc oxide et al., and traditional silver bactericide type. The photocatalyst type of bactericides has a passive bactericidal mechanism, requiring sunlight or UV ray radiation to produce bactericidal effect. The biggest shortcoming of photocatalyst type is that it decomposes fiber while it is decomposing bacteria. On the other side, silver bactericide type can actively interact with enzymes in bacteria or destroy cell walls to achieve bactericidal effect. Thus, the invention selects silver bactericides as the starting reactants for antibacterial textiles.

Since textile products require lightweight, thinness and softness in use, the inorganic bactericidal powders need very small diameter. With the development of nano-technology, presently there are various techniques to reduce the particle size to nano scale. Besides, nano-particles have apparently different characteristics from bulk materials. Adding nano-particles could enhance intrinsic material properties and develop different properties. The invention uses radiation technique to combine silver bactericides and polymer fibers to product lightweight antibacterial textiles.

References:

1. Hughes G., Radiation Chemistry, Oxford University Press, London, UK, (1973).

2. Hayashi S., K. Fujiki, N. Tsubokawa, Grafting of hyperbranched polymers onto ultrafine silica; postgraft polymerization of vinyl monomers initiated by pendant initiating groups of polymer chains grafted onto the surface, Reactive & Functional Polymers, 46, (2000) 193-201.

3. Hao-Tsi Lin, Use of Co-60 Radiation and UV Radiation to Modify the Hydrophilicity and Bactericidal Effect for Polypropylene Nonwovens with Acrylic Acid, N-isopropylacrylamide or Chitosan, Chang Gung University Ph.D. thesis (2003).

4. Maxwell A, Critchlow S. E. : Mode of Action, In Quinolone Antibacterials, Edited by Kuhlmann J., Dalhoff A., Zeiler H. J., Springer Verlag (1997), P. 119.

5. Subbalakshmi C. N. Sitaram: Mechanism of antimicrobaial action of indelicidin, FEMS Microbiol Letts, (1998), P. 91.

### SUMMARY OF THE INVENTION

The main objective for the invention is to use radiation technique to graft inorganic silver bactericides onto Nylon or PET fibers to produce antibacterial performance textiles.

Basically, nano-fabrication of antibacterial textiles is to use materials like silver nano-particles as bactericidal additive. Due to its excellent thermal stability, it can be added to fiber mother solution, which followed by spinning leads to producing antibacterial fibers. Since the diameter of nano-particles is very small and their surface activity is large, they tend to form aggregates under the high-temperature influence in the process and they are difficult to combine with fibers. It is necessary to develop a new technology to simplify the manufacturing process that will firmly fasten inorganic bactericidal nano-powders onto fibers in the back stage of the process.

The purpose of the invention is to use silver nano-compound as performance additive, which uses radiation modification to reduce and deposit silver particles onto PET or Nylon fiber surface. Such products, after SEM (scanning electronic microscope), ICP (inductively coupled plasma), XPS (X-ray photoelectron spectroscopy) and bactericidal test et al., have proved to have excellent antibacterial performance.

### DETAILED DESCRIPTION OF THE PREFERRED EMBODIMENT

The radiation technique for the invention is to use Co-60 γ-ray (or E-beam) radiation on fiber materials. It includes mutual irradiation process and pre-irradiation process, which has main steps as follows:

1. Mutual Irradiation Process

(a) Cut Nylon-6 or PET fabric into ≧20 cm×20 cm base material;

(b) Immerse Nylon-6 or PET base material in silver/silicon dioxide (Ag/SiO₂) or silver nitrate solution for over two hours;

(c) Take out the fabric material and press it over 2.0 kg/cm² under rollers to squeeze out excessive silver nitrate solution;

(d) Put Nylon-6 or PET fabric material into a plastic bag, such as PE ziplock bag, for radiation treatment.

2. Pre-Irradiation Process:

(a) Cut Nylon-6 or PET fabric into ≧20 cm×20 cm base material;

(b) Put Nylon-6 or PET fabric material into a plastic bag, such as PE ziplock bag, for radiation treatment;

(c) Immerse Nylon-6 or PET fabric material in silver/silicon dioxide (Ag/SiO₂) or silver nitrate solution for different immersion times. Take out the material to pressing treatment. Place the material in oven for fast drying.

### EXAMPLE 1

Take two different fabric materials in the same size (≧20×20 cm²). Immerse the Nylon or PET fabric material in a suspension solution of over 2.0 wt % Ag/SiO₂ bactericidal powers for over two hours. Use press to remove excessive liquid. Then we use radiation less than 100 kGy to combine bactericidal powders and fabric material. Clean the fabric material to obtain the antibacterial product. FIG. 1(a) and FIG. 1(b) are the SEM pictures for bactericidal nano-powders on Nylon or PET after radiation exposure. It is observed that fiber surface has many bactericidal nano-powders after drying, possibly either through van der Waals forces or interaction with fibers (like covalent bond).

Cleaning is usually thought to remove the bactericidal powders that are on fiber surface through van der Waals forces. Silver containing fibers after agitation, with fiber surface subject to collisions and shaking, for a long time shall have grafting between surface remaining bactericidal nano-powders and fibers. FIG. 2 clearly shows the remaining bactericidal powders are greatly reduced on the fiber surface of the cleaned fabric material. From the XRD graph for the antibacterial fabric, it is difficult to determine whether there is silver component. This might be because the fiber had very low content of silver on surface. XPS (X-ray photoelectron spectra) was used to determine whether the fiber has silver. FIG. 3 and FIG. 4 are the XPS diagrams for Nylon or PET that has bactericidal nano-powders of Ag/SiO₂ and Ag/SiO₂P (porous). From FIG. 3 and FIG. 4, it is found that the characteristic peak of Ag_{3d} exists at a binding energy of 368 eV, which indicates the produced antibacterial fabric contains silver.

To determine the silver content after radiation exposure, concentrate nitric acid was used to dissolve the silver in the nano-powders on the fabric (because silver reacts with nitric acid to form silver nitrate). Then ICP is used to quantitatively determine the silver content of antibacterial fabric. Table 1 is the powder content and silver content for Nylon or PET fabric after Co-60 irradiation of less than 100 kGy. From Table 1 it is clearly seen than the silver content is as high as 1 wt % before cleaning. But after cleaning, the silver content decreases a lot, which indicates only a small amount of bactericidal nano-powders remain on the fabric surface after cleaning. From Table 1, it is also PET fiber has much less silver content than Nylon fiber. By calculation, it is found SiO₂ from Ag/SiO₂ and Ag/SiO₂P SiO₂ remain 15 wt % and 16 wt % respectively on Nylon surface after cleaning. The amount of powders on PET surface is low. It is 5 wt % and 4 wt % from Ag/SiO₂ and Ag/SiO₂P respectively. Because the two bactericidal powders have relatively low silver content, Nylon or PET fabric has low silver content.

TABLE 1
SiO₂ Powder Content and Silver Content for Nylon or PET
Fabric after Co-60 Irradiation of less than 100 kGy

| | Before cleaning | After cleaning | |
|---|---|---|---|
| | Ag wt % | Ag wt % | SiO₂ wt % |
| Nylon | | | |
| Ag/SiO₂ | 1.09 wt % | 0.57 wt % | 15 wt % |
| Ag/SiO₂P | 2.21 wt % | 0.31 wt % | 16 wt % |
| PET | | | |
| Ag/SiO₂ | 1.22 wt % | 0.19 wt % | 5 wt % |
| Ag/SiO₂P | 0.22 wt % | 0.08 wt % | 4 wt % |

### EXAMPLE 2

The irradiation method uses radiation to directly reduce silver particles on fiber surface. First, cut Nylon or PET fabric material (≧20×20 cm ) to immerse in silver nitrate solution for over two hours. Squeeze out excessive liquid by pressing. Then proceed with radiation in less than 80 kGy. FIG. 5 and FIG. 6 are SEM pictures for Nylon or PET immersed in silver nitrate and exposed to radiation. FIG. 5(a) is the SEM picture for Nylon fabric before cleaning, which indicates many tiny particles on surface. Although there is a small amount of aggregates, generally the particle distribution is uniform. It is thought that after radiation the fiber surface should have reduced silver particles and deposits of silver nitrate. Further silver particles include surface silver particles attracted by van der Waals forces and bonded or chelated particles. The silver particles after cleaning should be those on the surface through chemical bonding or chelating. FIG. 5(b) is the SEM picture after cleaning, which indicates the particle distribution is smaller than that in FIG. 5(a). It also indicates most silver particles have been washed away and only bonded or chelated ones remain.

FIG. 6(a) the SEM pictures for PET immersed in silver nitrate and exposed to radiation. From the SEM pictures, it is found before cleaning PET fiber surface has silver distribution in tree-branch or granular deposits. After cleaning the tree-branch deposits are removed, but many tiny silver particles remain on fiber surface, as shown in FIG. 6(b). Besides, XPS (X-ray photoelectron spectra) is used to determine whether fiber has silver content. FIG. 7 and FIG. 8 are XPS spectrums for Nylon or PET fabric containing Ag and AgNO₃ bactericidal powders after direct radiation. From FIG. 7 and FIG. 8, it is found the characteristic peak of Ag_{3d} exists at a binding energy of 368 eV, which indicates the produced antibacterial fabric contains silver.

Table 2 shows the ICP measured amount of grafted silver on fiber surface before and after cleaning. For PET and Nylon fabrics immersed in over 0.25M silver nitrate solutions and pressed later, after cleaning the ICP measurement indicates silver content of 11.57 wt % and 15.33 wt % respectively. After water cleaning the silver content is greatly reduced to 1.08 wt % and 2.15 wt % respectively. This should be the silver content for those particles bonded to fiber surface. Besides, PET has much lower silver content than Nylon because PET fabric is hydrophobic and contains less water after pressing.

TABLE 2
ICP Measured Amount of Grafted Silver on
Fiber Surface before and after Cleaning

| | Before cleaning Wt % | After cleaning Wt % |
|---|---|---|
| PET | 11.57 | 1.08 |
| Nylon | 15.33 | 2.15 |

### EXAMPLE 3

Besides the above post-irradiation and direct-irradiation methods, the process method also studies the variation of silver content for Nylon or PET fabric under pre-irradiation. The process for pre-irradiation is described as follows: irradiate Nylon or PET fabric with y-ray less than 60 kGy and immerse it in silver nitrate solution for over 20 minutes; proceed with pressing, drying and cleaning to produce nano antibacterial textile.

Table 3 shows the amount of silver content for fabric after pre-irradiation. From the table, it is known that the pre-irradiated fabric after cleaning has low silver content, less than 0.1 wt %. There are two reasons: first, the free radicals or peroxides produced on Nylon or PET surface exist for a very short of time and fail to reduce the silver ions in silver nitrate solution; second, the reduced silver particles need hydrated electrons, but the pre-irradiation fails to provide sufficient hydrated electrons to effectively reduce the silver ions in the solution.

TABLE 3
Silver Content for Nylon and PET Fabrics through Pre-irradiation

| | | Before cleaning | After cleaning |
|---|---|---|---|
| | Sampling | Ag wt % | Ag wt % |
| Nylon | >0.25M AgNO₃ | 0.141 | 0.01 |
| PET | >0.25M AgNO₃ | 0.191 | 0.006 |

From the above results and bactericidal test, it is known that except the pre-irradiation effect is not clear, the remaining two methods have strong bactericidal effects. Especially the Nylon or PET fabric with silver from direct radiation and reduction process has significant bactericidal effect, with bactericidal power up to 99.0% (with respect to Staphylococcus aureus). The silver containing in PET bactericidal performance is better than that of Nylon.

### BRIEF DESCRIPTION OF THE DRAWINGS

FIG. 1 is the SEM pictures for (a) Nylon and (b) PET fibers containing Ag/SiO₂ powders after radiation exposure.

FIG. 2 is the SEM pictures for (a) Nylon and (b) PET fibers containing Ag/SiO₂ powders after radiation exposure and cleaning.

FIG. 3 is the XPS spectrums for Nylon fiber containing bactericidal powders of (a) Ag/SiO₂P and (b) Ag/SiO₂ after radiation exposure.

FIG. 4 is the XPS spectrums for PET fiber containing bactericidal powders of (a) Ag/SiO₂P and (b) Ag/SiO₂ after radiation exposure.

FIG. 5 is the SEM pictures for Nylon fiber immersed in silver nitrate solution and exposed by radiation; Figure (a) is before cleaning, Figure (b) is after cleaning.

FIG. 6 is the SEM pictures (magnification 2000 times) for PET fiber immersed in silver nitrate solution and exposed by radiation; Figure (a) is before cleaning, Figure (b) is after cleaning.

FIG. 7 is the XPS spectrums for Nylon fiber containing Ag bactericidal powders after direct radiation.

FIG. 8 is the XPS spectrums for PET fiber containing Ag bactericidal powders after direct radiation.

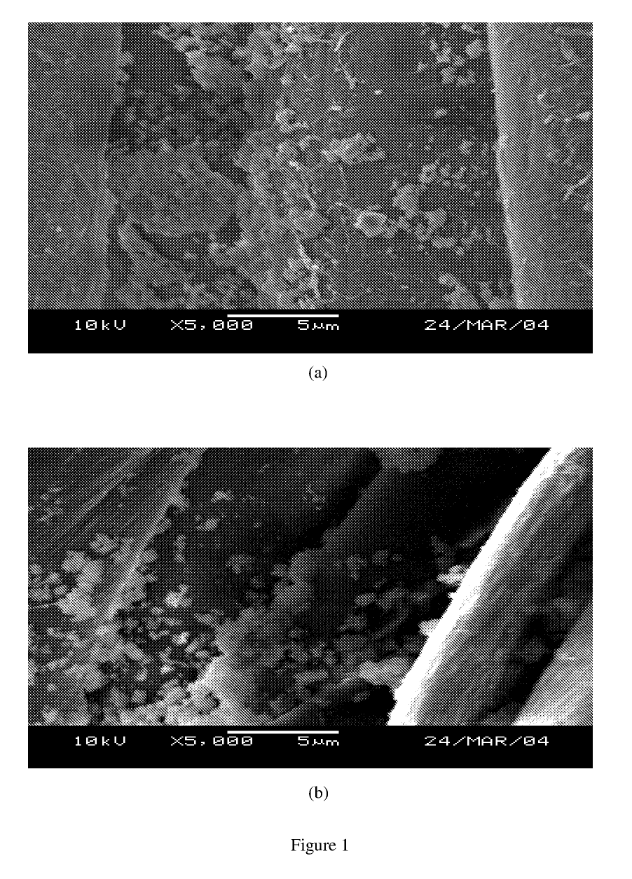

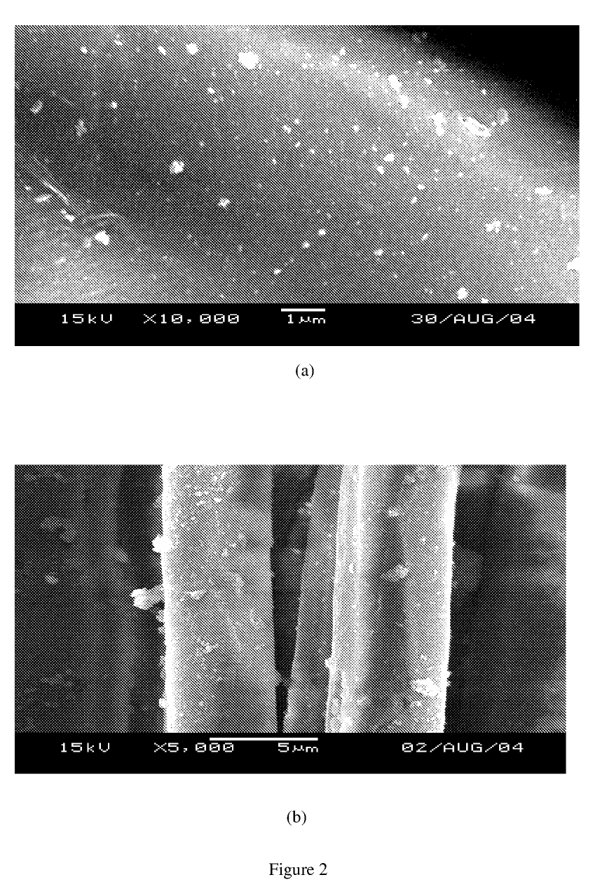

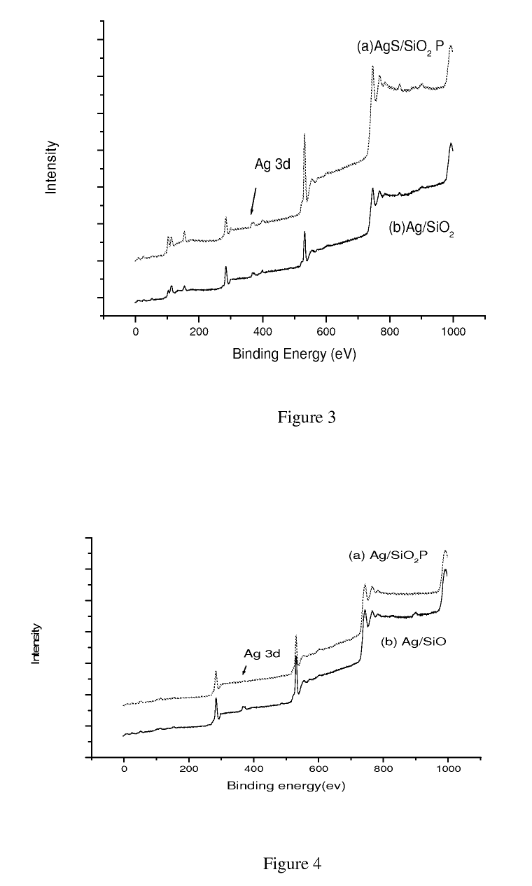

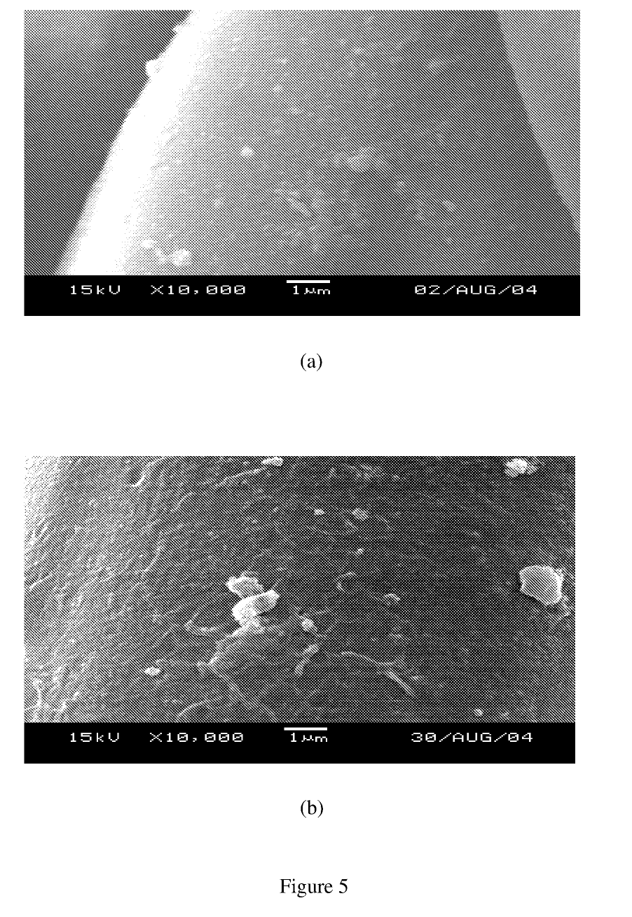

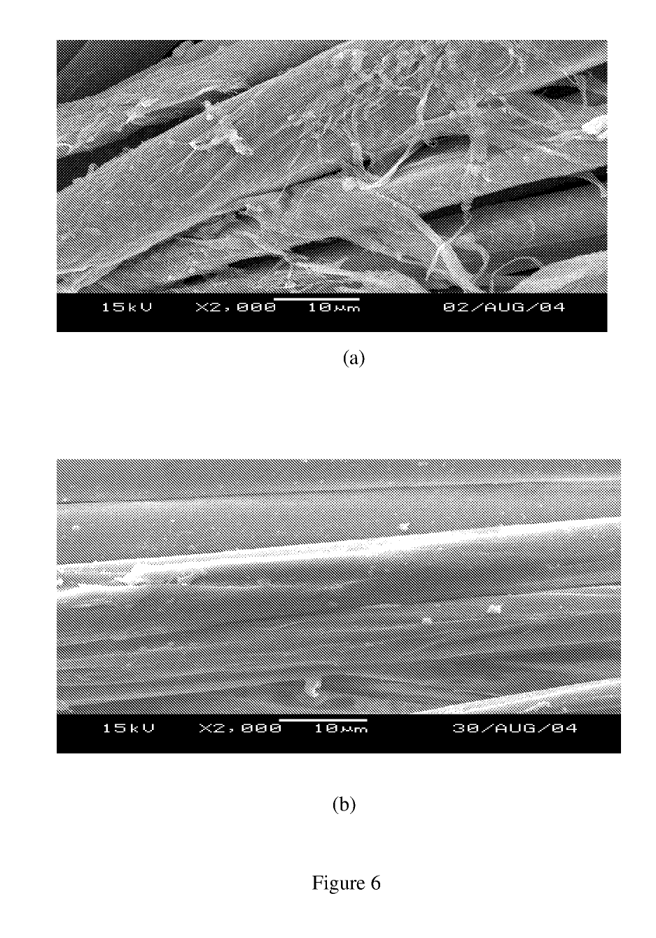

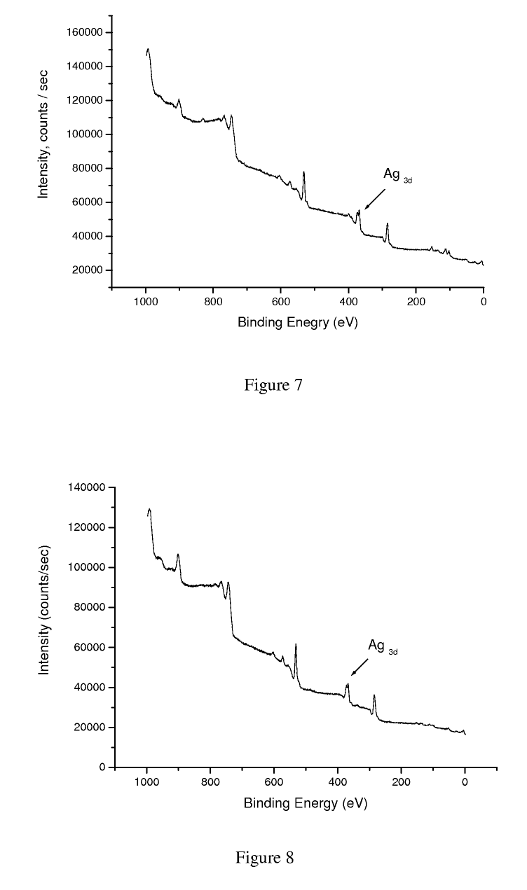

What is claimed is:

1. A process to make textile nano-composite containing silver compound includes:
(a) Cut Nylon-6 or PET fabric into ≧20 cm×20 cm base material;
(b) Immerse Nylon-6 or PET base material in silver/silicon dioxide (Ag/SiO₂) or silver nitrate solution for over two hours;
(c) Take out the fabric material and press it over 2.0 kg/cm under rollers to squeeze out excessive silver nitrate solution;
(d) Put Nylon-6 or PET fabric material into a plastic bag, such as PE ziplock bag, for radiation treatment.

2. As described in claim 1 for a process to make textile nano-composite containing silver compound, when the bactericidal powder is Ag/SiO₂ solution, the radiation is 5 kGy˜100 kGy.

3. As described in claim 1 for a process to make textile nano-composite containing silver compound, when the bactericidal powder is AgNO₃ solution, the radiation is 5 kGy˜80 kGy.

4. As described in claim 1 for a process to make textile nano-composite containing silver compound, the radiation uses Co-60 γ-ray.

5. As described in claim 1 for a process to make textile nano-composite containing silver compound, the radiation can use electron beam.

6. As described in claim 1 for a process to make textile nano-composite containing silver compound, the amount of grafted bactericidal powders onto Nylon or PET can be up to 15 wt % and 5 wt % respectively, both with silver content less than 1 wt %.

7. As described in claim 1 for a process to make textile nano-composite containing silver compound, the bactericidal powders are Ag/SiO2 or silver metal compound that after radiation are deposited as granules on fiber surface and may form aggregates in a small portion.

8. As described in claim 1 for a process to make textile nano-composite containing silver compound, the produced polyester composite has over 99.0% bactericidal power.
